(19) United States
(12) Patent Application Publication
(10) Pub. No.: US 2024/0207327 A1
(43) Pub. Date: Jun. 27, 2024

(54) COMPOSITION INCLUDING PROPOLIS AND SORBUS COMMIXTA AS ACTIVE INGREDIENTS FOR ALLEVIATING AND TREATING INFLAMMATORY DISEASES AND ALLERGIC DISEASES

(71) Applicant: Andong National University Industry-Academic Cooperation Foundation, Andong-si, Gyeongsangbuk-do (KR)

(72) Inventors: Jae Hwan LIM, Gwacheon-si (KR); Chan Gun YUN, Andong-si (KR); Se Hee CHO, Andong-si (KR); Jee-In KIM, Andong-si, Gyeongsangbuk-do (KR); Sang Ryul NAM, Gyeongsangbuk-do (KR); Sung Min JEONG, Andong-si, Gyeongsangbuk-do (KR)

(73) Assignee: Andong National University Industry-Academic Cooperation Foundation, Andong-si, Gyeongsangbuk-do (KR)

(21) Appl. No.: 18/544,358

(22) Filed: Dec. 18, 2023

(30) Foreign Application Priority Data
Dec. 22, 2022 (KR) 10-2022-0181414

(51) Int. Cl. A61K 35/644; A61K 31/352; A61K 36/73; A61P 29/00; A61P 37/08

(52) U.S. Cl. CPC A61K 35/644; A61K 31/352; A61K 36/73; A61P 29/00; A61P 37/08; A61K 2236/333

(57) ABSTRACT

Proposed are pharmaceutical and food compositions for alleviating and treating inflammatory diseases and allergic diseases, the compositions including propolis and Sorbus commixta as active ingredients. Preparation methods of the compositions are also proposed. The compositions are prepared by extracting a propolis ethanolic extract from a propolis powder using an aqueous ethanol solution through extraction and filtration, extracting a Sorbus commixta ethanolic extract from a Sorbus commixta powder using an aqueous ethanol solution through extraction and filtration, and mixing the propolis ethanolic extract and the Sorbus commixta ethanolic extract. Since propolis and Sorbus commixta are contained in the pharmaceutical and food compositions, the pharmaceutical and food compositions exhibit excellent anti-inflammatory and anti-allergic effects.

### CROSS REFERENCE TO RELATED APPLICATION

The present application claims priority to Korean Patent Application No. 10-2022-0181414, filed Dec. 22, 2022, the entire contents of which is incorporated herein for all purposes by this reference.

### BACKGROUND OF THE DISCLOSURE

### 1. Field of the Disclosure

The present disclosure relates to a composition including propolis and Sorbus commixta as active ingredients for alleviating or treating inflammatory diseases and allergic diseases. More particularly, the present disclosure relates to a composition including propolis and Sorbus commixta as active ingredients for alleviating or treating inflammatory diseases and allergic diseases, and a preparation method thereof, in which the propolis is selected and obtained from a specific production region which produces propolis with better alleviation or treatment effects than propolis produced from other production regions since the efficacy of propolis varies depending on the content of the flavonoid components because the content of the flavonoid components in propolis varies from region to region, and the obtained propolis is used in combination with Sorbus commixta.

### 2 Description of the Related Art

According to a recent disease burden study, the inflammatory response refers to the occurrence of a series of complex physiological reactions triggered by injury or foreign agents such as bacteria, fungi, and viruses, the inflammatory response including enzyme activation by various inflammatory mediators and immune cells, secretion of inflammatory mediators, fluid infiltration, cell migration, and tissue destruction. The inflammatory response is accompanied by symptoms such as erythema, edema, fever, pain, and so on. The inflammatory response restores a body function by removing external infectious sources and regenerating damaged tissues, but when antigens are not removed or when internal substances are a major cause of the response, which results in an excessive or continuous inflammatory response, it may rather cause mucosal damage and tissue destruction, and may even lead to diseases such as cancer, inflammatory skin disease, inflammatory bowel disease, and arthritis.

Until now, antihistamines, vitamin ointments, and corticosteroids have been mainly used to treat inflammatory diseases such as the above, but those drugs are often temporary in effect, and have severe side effects in many cases, so there is a growing voice of developing new substances with therapeutic effects for inflammatory diseases, and recently, a growing attention is paid to anti-inflammatory drugs with minimal side effects by isolating compounds with strong anti-inflammatory effects from natural products.

Allergies develop in the form of atopic dermatitis, bronchial asthma, allergic rhinitis, allergic keratitis, and skin urticaria in a human body, and mast cells are known to play a primary role. In the later stages of the allergic diseases, inflammatory cells begin to infiltrate the tissues, so anti-inflammatory medications are administered in conjunction with antihistamines in the treatment of the allergic diseases.

Currently, commonly available allergy treatments include antihistamines, steroidal or non-steroidal anti-inflammatory medications, which are primarily effective against exogenous pathogens, and are known not to be effective to treat the underlying cause of the allergies that ease an excessive humoral immunity or suppress an IgE production.

Against this backdrop, many studies have recently been published in order to alleviate the allergic symptoms by using natural products.

Likewise, the natural products are compounds or metabolites obtained in nature such as plants, minerals, animals, and microorganisms, and herbal medicines are those used in an essentially unchanged state through simple processing such as cutting, grinding, or extraction to use the natural products or in a dosage form of medicinal ingredients.

Most of the active ingredients in the natural products contain unknown compounds, making it difficult to clearly identify each ingredient, but active research is underway in many aspects to determine the efficacy of the various ingredients. Research on botanicals, which make up more than 80% of the herbal medicines, has reported that the various herbal medicines have various efficacies such as antioxidant, anti-inflammatory, anti-cancer, and antiviral effects.

Sorbus commixta, an example of the botanicals has been reported to have anti-inflammatory, anti-cancer, and antioxidant effects.

Propolis is a compound made of a resinous substance that bees collect from parts of plants, such as tree buds and plant sap, plus pollen and bee secretions, and is known to be used by bees to seal their hives to maintain temperature and humidity, and to protect the hives from pathogens and viruses.

Propolis is composed of resin, beeswax, essential oils, pollen, and organic compounds. Propolis has been widely used as a medicine so propolis has been called a natural antibiotic. In modern times, the efficacies of propolis have been studied extensively, and have been reported to have various effects such as antibacterial, anti-cancer, anti-inflammatory, and antiviral effects. This is likely due to the various flavonoid components that propolis contains, which has led to the development of various medicines and supplements using propolis.

The flavonoid components, which are paid attention to as the main active ingredients in propolis, are phytochemicals that are naturally occurring chemicals found in almost all plants, primarily vegetables, greens, and fruits. The reason why the flavonoid components usually found in plants can be found in propolis, which is not a plant, in the form of protocorm, is because bees make propolis from a resin collected from tree buds and sap in surrounding vegetation.

Accordingly, the content of flavonoid components as active ingredients in propolis varies, depending on propolis production regions because vegetation and environmental conditions in the regions differ. Thus, there are few universal standards or standardized studies on the efficacy of propolis, and it is difficult to compare efficacies of propolis produced from different regions.

### Documents of Related Art

### Patent Document

(Patent Document 0001) Korean Patent Application Publication No. 10-2012-0120515 titled “Composition and Manufacturing Method of Functional Food Containing Propolis and Herbal Extract Which Has Antimicrobial, Antioxidant, and Immune-Stimulating Activity”
(Patent Document 0002) Korean Patent No. 10-0922987 titled “A Pharmaceutical Composition Having Anti-Inflammatory, Analgesic, Antipyretic, Antioxidant, and Antihistamine Effect, and a Method for Preparation Thereof”
(Patent Document 0003) Korean Patent No. 10-2001024 titled “Composition for Anti-Inflammatory and Anti-Helicobacter Pylori Comprising Propolis”

### SUMMARY OF THE DISCLOSURE

The present disclosure aims to provide a pharmaceutical composition or food composition including propolis and Sorbus commixta as active ingredients for alleviating or treating inflammatory diseases and allergic diseases, in which the propolis is selected and obtained from a specific production region which produces propolis with better alleviation or treatment effects than other regions given that the efficacy of propolis varies depending on the content of the flavonoid components, and the content of the flavonoid components in propolis varies from region to region.

To achieve the above objective, the present disclosure provides a method of preparing a pharmaceutical composition or food composition for alleviating or treating inflammatory diseases and allergic diseases, the method including: a first step of preparing a propolis ethanolic extract by subjecting a propolis powder to an extraction process using an aqueous ethanol solution, followed by filtering; a second step of preparing a Sorbus commixta ethanolic extract by subjecting a Sorbus commixta powder to an extraction process using an aqueous ethanol solution, followed by filtering; and a third step of mixing the propolis ethanolic extract resulting from the first step and the Sorbus commixta ethanolic extract resulting from the second step, thereby producing the pharmaceutical composition or food composition for alleviating and treating inflammatory diseases and allergic diseases.

In addition, the propolis ethanolic extract resulting from the first step contains 0.5 to 2.5 μg/mL of Quercetin, 1 to 5 μg/mL of Kaempferol, 1 to 2.5 μg/mL of Chrysin among the flavonoid components.

According to the present disclosure, the pharmaceutical composition or food composition for alleviating or treating inflammatory diseases and allergic diseases includes propolis and Sorbus commixta as active ingredients due to better anti-inflammatory and anti-allergic effects thereof, whereby the pharmaceutical composition or food composition exhibits good anti-inflammatory and anti-allergic effects.

### BRIEF DESCRIPTION OF THE DRAWINGS

FIG. 1 is a view illustrating a process of preparing a pharmaceutical composition including propolis and Sorbus commixta as active ingredients for alleviating or treating inflammatory diseases and allergic diseases;

FIG. 2 is a graph illustrating an HPLC analysis result of regional propolis, in which Cr, Ca, Co, Ga, Ka, Pi, and Qu are acronyms of ingredient names representing Chrysin, Caffeic acid, p-Coumaric acid (4-Hydroxycinnamic acid), Galangin, Kaempferol, Pinocembrin, and Quercetin, respectively; and Sc, Jd, Mw, Ws, Cp, Gs, Go, Ad, Bh, Ms, Gw, and Gwi are region names representing Sancheong, Jangdeung-dong, Mangwol-dong, Wolsan-myeon, Changpyeong-myeon, Go Seomyeon, Andong, Bonghwa, Masan, and Gunwi, respectively;

FIG. 3 is a graph illustrating a flavonoid content in each regional propolis extract, in which Cr, Ca, Co, Ga, Ka, Pi, and Qu are acronyms of ingredient names representing Chrysin, Caffeic acid, p-Coumaric acid (4-Hydroxycinnamic acid), Galangin, Kaempferol, Pinocembrin, and Quercetin, respectively; and Sc, Jd, Mw, Ws, Cp, Gs, Go, Ad, Bh, Ms, Gw, and Gwi are region names representing Sancheong, Jangdeung-dong, Mangwol-dong, Wolsan-myeon, Changpyeong-myeon, Go Seomyeon, Andong, Bonghwa, Masan, and Gunwi, respectively;

FIG. 4 is a graph illustrating the cell viability of PAW 264.7 cell line when the cell line is treated with different components of flavonoids and Sorbus commixta ethanol extract (SEE), in which the used flavonoid components include Pinocembrin, Quercetin, Kaempferol, Galangin, Chrysin, Caffeic acid, and p-Coumaric acid (4-Hydroxycinnamic acid), and SEE represents Sorbus commixta ethanol extract;

FIG. 5 is a graph illustrating the amounts of nitrite oxide (NO) produced when treated with different components of flavonoids and different concentration levels of Sorbus commixta ethanol extract (SEE), in which the used flavonoid components include Pinocembrin, Quercetin, Kaempferol, Galangin, Chrysin, Caffeic acid, and p-Coumaric acid (4-Hydroxycinnamic acid), and SEE represents Sorbus commixta ethanol extract;

FIG. 6 is a graph illustrating the amounts of nitrite oxide (NO) produced when combinedly treated with different components of flavonoids and Sorbus ethanol extract (SEE), in which the used flavonoid components include Quercetin, Kaempferol, and Chrysin, and SEE represents Sorbus commixta ethanol extract;

FIG. 7 is a graph illustrating the cell viability of RBL-2H3 cell line when treated with different components of flavonoids and different concentration levels of Sorbus commixta ethanol extract (SEE), in which the used flavonoid components include Quercetin, Kaempferol, and Chrysin, and SEE represents Sorbus commixta ethanol extract;

FIG. 8 shows a graph illustrating the degranulation of RBL-2H3 cell line (associated with allergenicity) when treated with different components of flavonoids and different concentration levels of Sorbus commixta ethanol extract (SEE), the used flavonoid components include Quercetin, Kaempferol, and Chrysin, and SEE represents Sorbus commixta ethanol extract;

FIG. 9 is a graph illustrating the degranulation of RBL-2H3 cell line (associated with allergenicity) when combinedly treated with different components of flavonoids and Sorbus ethanol extract (SEE), the used flavonoid components include Quercetin, Kaempferol, and Chrysin, and SEE: Sorbus commixta ethanol extract.

### DESCRIPTION OF THE PREFERRED EMBODIMENTS

Hereinafter, preferred embodiments and experimental examples of the present disclosure will be described in detail with reference to the accompanying drawings, and a detailed description of known functions and configurations that may unnecessarily obscure the gist of the present disclosure will be omitted.

The term “inflammatory disease”, as used herein, refers to a disease caused by inflammatory substances (inflammatory cytokines) such as NO, and the inflammatory substances cause an immune system overreaction in response to a harmful stimulus such as an inflammatory agent or irradiation.

Herein, the inflammatory disease may be any one selected from the group consisting of insulin-dependent diabetes, eczema, allergy, atopic dermatitis, acne, atopic rhinitis, pneumonia, allergic dermatitis, chronic sinusitis, contact dermatitis, seborrheic dermatitis, Gastritis, gout, gouty arthritis, ulcers, chronic bronchitis, ulcerative colitis, ankylosing spondylitis, sepsis, vasculitis, bursitis, temporal arteritis, solid tumors, Alzheimer's disease, atherosclerosis, obesity, viral infections, and nonalcoholic steatohepatitis, but is not limited thereto.

The term “allergy”, as used herein, refers to a phenomenon where a living body in contact with a foreign substance exhibits a different reaction than normal to that substance.

Herein, the allergic disease may be any one selected from the group consisting of allergic rhinitis, allergic conjunctivitis, allergic asthma, allergic dermatitis, allergic bronchopulmonary aspergillosis, and allergic stomatitis but is not limited thereto.

The term “alleviation”, as used herein, refers to any action that suppresses or delays a progression of allergic diseases by administration of the compositions of the present disclosure.

The term “treatment”, as used herein, refers to any activity in which symptoms of allergic diseases are alleviated or beneficially changed by administration of the compositions of the present disclosure.

The term “administration”, as used herein, means providing a given composition of the present disclosure to a subject by any suitable method.

Hereinafter, the present disclosure relates to a composition including propolis and Sorbus commixta as active ingredients for alleviating or treating inflammatory diseases and allergic diseases with, and a preparation method thereof, and the present disclosure will be described in detail through embodiments.

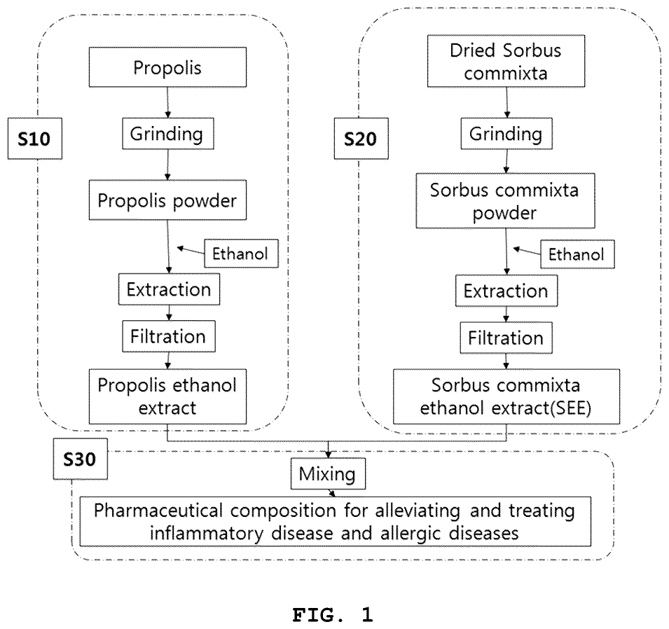

Specifically, as shown in FIG. 1, the preparation method includes a propolis ethanol extract preparation step S10, a Sorbus commixta ethanol extract preparation step S20, and a mixture preparation step S30. The preparation method has an advantage of providing an anti-inflammatory and anti-allergic pharmaceutical composition or food composition that exhibits excellent anti-inflammatory and anti-allergic effects by including propolis and Sorbus commixta having excellent anti-inflammatory and anti-allergic effects as active ingredients. More details thereof will be described below.

### <Method of Preparing Pharmaceutical Composition for Alleviating or Treating Inflammatory Diseases and Allergic Diseases, According to Present Disclosure>

### 1. First Step S10: Preparation of Propolis Ethanolic Extract

In this step, a propolis powder is subjected to an extraction process using an aqueous ethanol solution, followed by filtering. As a result, a propolis ethanolic extract is obtained.

For reference, propolis is made from a mixture of tree secretions collected by bees, bee saliva, and beeswax, and has been reported to have a variety of effects, including antibacterial, anti-cancer, anti-inflammatory, and antiviral properties. Flavonoids are a class of phytochemicals that are naturally occurring in plants and have recently gained prominence.

The flavonoid components contained in propolis vary somewhat depending on the propolis production regions, and the content of flavonoid components in propolis varies depending on extraction methods.

Therefore, in this step, to maximize the extraction of the flavonoid components contained in propolis, a propolis powder is added with a 90% to 95% (v/v) ethanol aqueous solution, in which the amount of the ethanol aqueous solution added is 2 to 3 times the amount of the propolis powder, and extraction was performed at a temperature of 35° C. to 37° C. at a speed of 100 to 200 rpm for 20 to 25 hours to prepare a flavonoid ethanol extract, which is used as an active ingredient in the composition of the present disclosure.

In this case, when the concentration of the ethanol aqueous solution is lower than 90% (v/v), the flavonoid components will not be sufficiently extracted. On the other hand, when the concentration of the ethanol aqueous solution is higher than 95% (v/v), there is a risk of the decomposition of some flavonoid components.

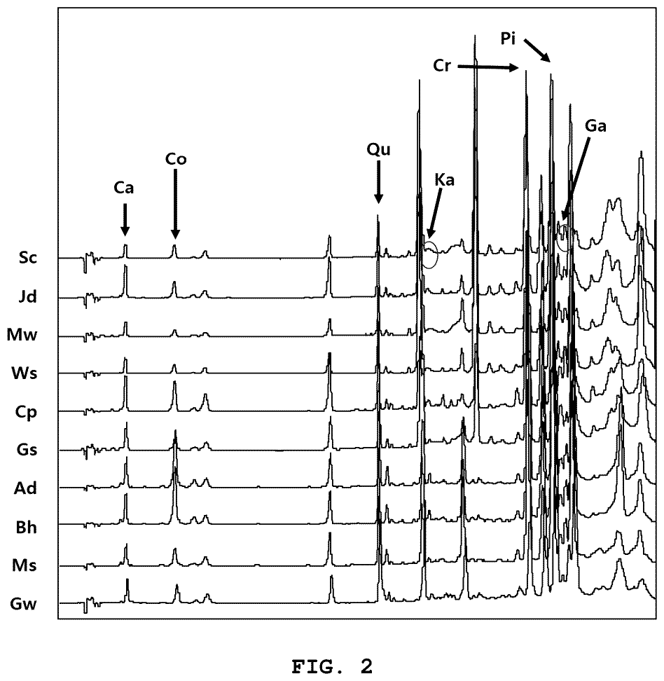

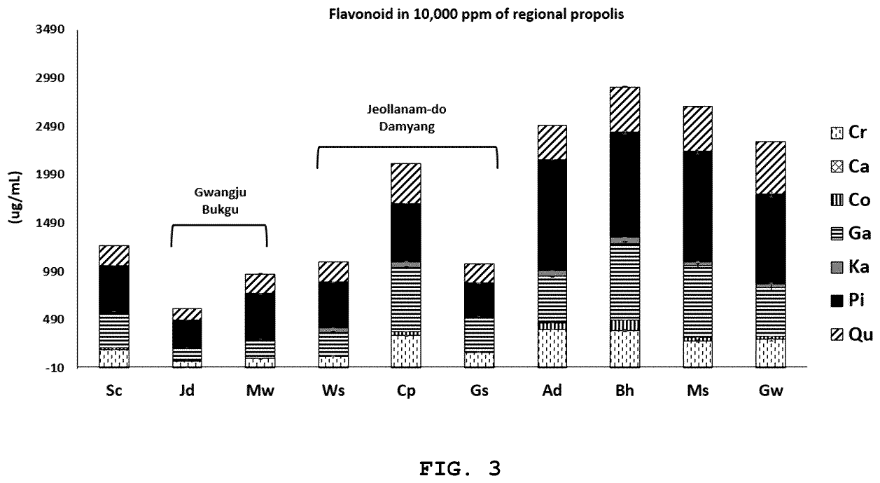

As shown in FIG. 2, the propolis ethanol extract thus obtained contains Pinocembrin, Quercetin, Kaempferol, Galangin, and Chrysin, Caffeic acid, and 4-Hydroxycinnamic acid (p-Coumaric acid) among many flavonoid components, and as shown in FIG. 3, the content of the flavonoid components in propolis considerably varies from region to region.

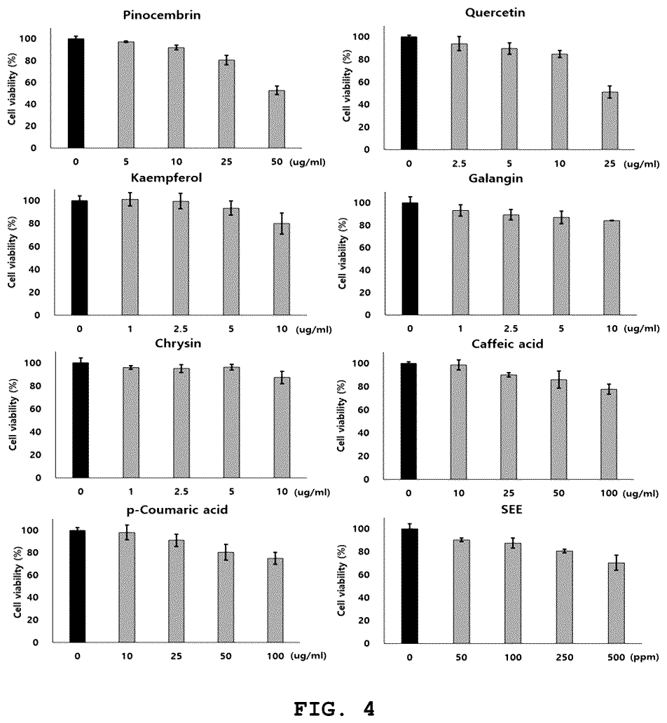

In addition, as shown in FIGS. 4 to 9, among the flavonoid components, Quercetin, Galangin, and Chrysin were found to be contained in the largest amount. However, in terms of the production of nitrite oxide (NO), the groups treated with Quercetin, Kaempferol, or Chrysin exhibited the highest inhibitory effect on NO production.

In particular, when the degranulation of RBL-2H3 cells is induced by antibody-antigen binding, beta-Hexosaminidase, which is an indicator of early allergic reactions, is released. The inventors confirmed that treatment with any one of the three flavonoid components (Quercetin, Kaempferol, and Chrysin) decreased the degree of degranulation of RBL-2H3 cells and identified herbal extracts that can show synergistic effects with the flavonoid components as shown below.

2. Second stepS20: Preparation of Sorbus commixta Ethanolic Extract

In this step, a Sorbus commixta powder is subjected to an extraction process using an aqueous ethanol solution, followed by filtering.

For reference, Sorbus commixta is a deciduous broad-leaved small tree in the Rosaceae family, mainly distributed in deep mountain foothills at an altitude of 500-1200 meters in Korea, China, and Japan. The bark of Sorbus commixta is called Ma-api, and has been used in oriental medicine to protect kidneys, and to treat bronchitis, severe gastritis, and bone pain, and the fruit of Sorbus commixta has been known to have anti-nervousness effects. The physiological activities of Sorbus commixta include suppression on diabetes and obesity, antioxidant and anti-photoaging effects, vascular inflammation suppression, and anti-atherosclerosis, and the components of Sorbus commixta include Neosakuranin, Lupeol, and Lupenone.

Therefore, in this step, in order to maximize the extraction of the active components contained in Sorbus commixta, a Sorbus commixta powder is added with a 70-80% (v/v) ethanol aqueous solution, in which the amount of the ethanol aqueous solution added is 10 to 12 times the amount of Sorbus commixta powder, and extraction was performed under conditions of 35° C. to 37° C. and 100 to 200 rpm for 20 to 25 hours to prepare a Sorbus commixta ethanol extract, which is used as an active ingredient in the composition of the present disclosure. In this case, when the concentration of the ethanol aqueous solution is less than 70% (v/v), the active components will not be extracted properly. On the other hand, when the concentration of the ethanol aqueous solution is greater than 80% (v/v), there is a risk of the decomposition of some active components.

As shown in FIGS. 4 to 9, cell viability, NO production ability, and degranulation of the Sorbus commixta ethanol extract thus obtained at concentrations of 25 to 100 ppm were identified. Therefore, the Sorbus commixta ethanol is applied to the present disclosure as an active ingredient whereby the Sorbus commixta ethanol extract is also dried and used in a powder form.

### 3. Third Step S30: Preparation of Pharmaceutical Composition or Food Composition for Alleviating or Treating Inflammatory Diseases and Allergic Diseases

In this step, a mixture of the propolis ethanolic extract resulting from Step 1 and the Sorbus commixta ethanolic extract resulting from Step 2 is prepared to produce a pharmaceutical composition or food composition for alleviating or treating inflammatory diseases and allergic diseases.

For reference, as shown in FIGS. 4 to 9, it is found that each of the three flavonoid components in the propolis ethanol extract had a different effect when it is solely used for treatment and when it is used in combination with the Sorbus commixta ethanol extract for treatment.

In other words, Quercetin, Kaempferol, and Chrysin exhibited a higher cell viability, a higher NO production ability, and a lower degranulation when each of them is used solely while the Sorbus commixta ethanol extract exhibits a higher degranulation when it is used solely.

However, when two substances (flavonoid components+the Sorbus commixta ethanol extract) were mixed in use, the NO production was significantly higher, and the degranulation degree was significantly lower than in the case where each of them was used solely, due to synergistic effects.

In particular, when the Quercetin is contained at a concentration of 0.5 to 2.5 μg/mL, the Kaempferol at a concentration of 1 to 5 μg/mL, the Chrysin at a concentration of 1 to 2.5 μg/mL, and the Sorbus commixta ethanol extract at a concentration of 25 to 100 ppm, the degree of degranulation was very low, and the NO production and rotaviral activity were high. It means that the composition containing the mentioned substances at the mentioned concentrations may exhibit efficacy in alleviation or treatment of inflammatory diseases and allergic diseases. More preferably, when the propolis containing Quercetin, Kaempferol, or Chrysin was mixed with the Sorbus commixta ethanol extract in a ratio of 1 to 5:25 by weight, the alleviating and therapeutic effects on inflammatory diseases and allergic diseases aremost suitable. On the other hand, when the content of the propolis isoutside the range, that is, the ratio of the propolis with respect to the Sorbus commixta ethanol extract is less than 1:25 by weight or exceeds 5:25 by weight, the synergistic effects on inflammatory diseases and allergic diseases is reduced.

The mixture almost does not exhibittoxicity or side effects and is thus used as an active ingredient in a pharmaceutical composition for the alleviation or treatment of allergic diseases, and may be safely used for long-term intake for alleviation purposes.

The pharmaceutical composition may also contain other pharmaceutically active ingredients than the ingredients or may contain other active ingredients. The pharmaceutical composition may be administered to a patient by any means, either orally or parenterally (e.g., intravenously, subcutaneously, intraperitoneally, or topically). Although there are no specific limitations on dosage form to be administered, the dosage formmay be determined by age, gender, or severity of the patient's condition.

Herein, a solid dosage form for oral administration may be an acid, a granule, a tablet, a capsule, a soft capsule, a pill, and the like. A liquid dosage form for oral administration may be asuspension, a solution, an emulsion, a syrup, an aerosol, and the like. A dosage form for parenteral administration may be an external preparation such as an acid, a granule, a tablet, a capsule, a sterile aqueous solution, a liquid, a non-aqueous solution, a suspension, an emulsion, a syrup, a suppository, or an aerosol, each being prepared according to conventional methods. Alternatively, the composition may be formulated in the form of a sterile injectable preparation prepared according to conventional methods.

The pharmaceutical composition may further contain conventionally used excipients, disintegrating agents, sweeteners, lubricants, flavoring agents, and so on. Examples of the disintegrating agent may includesodium starch glycolate, crospovidone, alginic acid, carboxymethyl cellulose calcium, carboxymethyl cellulose sodium), chitosan, guar gum, low-substituted hydroxypropyl cellulose, magnesium aluminum silicate, and so on.

In addition, the pharmaceutical composition may further include pharmaceutically acceptable additives. Examples of the pharmaceutically acceptable additives may include but are not limited to, starch, gelatinized starch, microcrystalline cellulose, lactose, povidone, colloidal silicon dioxide, calcium hydrogen phosphate, and lactose, mannitol, arabic gum, hydroxypropyl cellulose, sodium starch glycolate, carbauba wax, stearic acid, magnesium stearate, aluminum stearate, calcium stearate, white sugar, waffy, dextrose, sorbitol, and talc. The pharmaceutically acceptable additives may be included in an amount of about 0.1 to about 90 parts by weight based on the total weight of the pharmaceutical composition.

The food composition of the present disclosure may be formulated in the same way as the pharmaceutical composition. The food composition may be provided as an independent health functional food or may be used as a food additive. The food composition may be added to foods such as beverages, meat, chocolate, foods, confectionery, pizza, ramen, other noodles, gum, candy, ice cream, alcoholic beverages, vitamin complexes, health supplements, and so on.

In addition to the active ingredients of the present disclosure, the food composition may contain various nutrients, vitamins, minerals (electrolytes), flavoring agents such as synthetic and natural flavoring agents, colorants and thickening agents (cheese, chocolate, etc.), pectic acids, pectic salts, alginic acids and alginic salts, organic acids, protective colloidal thickeners, pH adjusters, stabilizers, preservatives, glycerin, alcohol, carbonating agents used in carbonated beverages, and so on. In addition, the food composition of the present disclosure may contain the flesh of fruit for the production of natural fruit juice, fruit juice drinks, and vegetable drinks.

The health functional food of the present disclosure may be prepared or processed into tablets, capsules, powders, granules, liquids, pills, and the like.

In the present disclosure, the term “health functional food” refers to a food manufactured by processing raw materials or ingredients having functionality useful to a human body in accordance with the Health Functional Food Act, and refers to a food to be taken in for the purpose of obtaining useful effects for health, such as regulation of nutrients or physiological effects for the structure and function of the human body.

The health functional food of the present disclosure may contain common food additives, and its suitability as a food additive is determined according to the General Rules and General Test Methods of the Korea Food Additives Code approved by the Ministry of Food and Drug Safety, unless otherwise specified. The determination is specifically made according to the relevant specifications and standards.

The items listed in the Korea Food Additives Code, for example, include chemical compounds such as ketones, glycine, calcium citrate, nicotinic acid, and cinnamic acid; natural additives such as dark pigment, licorice extract, crystalline cellulose, high-quality pigment, and guar gum; mixed preparations such as L-glutamate sodium preparations, noodle additive alkaline preparations, preservative preparations, and tar coloring preparations.

For example, the health functional foods in the form of a tablet are made by granulating a mixture of the active ingredients of the present disclosure with excipients, binders, disintegrating agents, and other additives in a conventional manner, then being added with lubricants for compression molding. The mixture may also be directly compressed for molding. The health functional foods in the form of a tablet may also contain a flavor enhancer or the like, as needed.

Among health functional foods in the form of capsules, hard capsules can be prepared by filling conventional hard capsules with a mixture of the active ingredients of the present disclosure mixed with additives such as excipients, and soft capsules can be prepared by filling a mixture of the active ingredients of the present disclosure mixed with additives such as excipients into a capsule base such as gelatin. The soft capsules may contain plasticizers such as glycerin or sorbitol, colorants, preservatives, and the like, as needed.

The health functional foods in the form of a pill may be prepared by molding a mixture of the active ingredients of the present disclosure with excipients, binders, disintegrants, and the like by conventional methods known in the art, and may be exfoliated with white sugar or other exfoliating agents as needed, or the surface of the pills may be coated with a substance such as starch or talc.

Granular health functional foods may be prepared by mixing the active ingredients of the present disclosure with excipients, binders, and disintegrants in granular form by conventional methods known in the art, and may contain flavoring agents, flavor enhancers, and the like, as needed.

The health functional foods may be beverages, meat, chocolate, confectionery food, pizza, ramen, other noodles, chewing gum, candy, ice cream, alcoholic beverages, vitamin complexes, and dietary supplements.

Hereinafter, the present disclosure will be described in more detail with reference to embodiments and experimental examples, but these embodiments and experimental examples are for illustrative purposes only and are not intended to limit the scope of protection of the present disclosure.

### <Example 1> Manufacturing Propolis Ethanolic Extract and Sorbus commixta Ethanolic Extract

Regional propolis was obtained from BEE HAPPY Cooperatives (Gunwi, Korea), and powdered using a grinder. The powdered propolis was subjected to an extraction process and was added with a 95% (v/v) Et-OH, in which the volume of the Et-OH is 2 times the volume of the powdered propolis, and the extraction was performed under conditions of 37° C. and 200 rpm for 24 hours in a shaking incubator, followed by filtering using 11 μm filter paper to prepare a propolis ethanol extract for use.

Sorbus commixta was supplied by Dongwoodang Pharmacy Co. and was extracted as follows.

That is, dried Sorbus commixta was powdered using a grinder. The dried Sorbus commixta was subjected to an extraction process and added with a 70% (v/v) Et-OH, in which the volume of the Et-OH is 10 times the volume of the dried Sorbus commixta. The extraction was performed under conditions of 37° C. and 200 rpm for 24 hours in a shaking incubator.

The extract was filtered using 11-μm filter paper to prepare a Sorbus commixta ethanol extract (SEE).

### <Experimental Example 1> HPLC Analysis of Flavonoid Components Contained in Propolis

### 1. Experiment Method

Experiments were carried out using HPLC (Perkin Elmer Inc., USA) for quantitative analysis of the flavonoid components in regional propolis. The propolis ethanol extract was diluted at a ratio of 1:100, filtered through a 0.45 μm filter, and was used for analysis. As standards, quercetin, kaempferol, galangin, chrysin, caffeic acid, p-coumaric acid (4-Hydroxycinnamic acid) (Sigma-Aldrich Inc, USA) and pinocembrin (Phytolab Inc., Germany) were purchased and used for the qualitative analysis of the components in propolis, and quantitative curves were drawn for the quantitative analysis of each flavonoid component.

For analysis, a Quasar™ C18 (150 mm×4.6 mm, 5 μm) column with 0.1% acetic acid (in water, solvent A) and acetonitrile (solvent B) was used, with varying ratios of mobile phases over time (0.0-10.0 min 80% A:20% B; 10.0-20.0 min 60% A:40% B; 20.0-35.0 min 50% A:50% B; 35.0-40.0 min 80% A:20% B) under the following conditions.

TABLE 1
Conditions

| | |
|---|---|
| Column | Quasar ™ C18 (150 mm × 4.6 mm, 5 μm) |
| Flow rate | 1 ml/min |
| Detection wavelength | 290 nm |
| Solvent A | 0.1% acetic acid in water |
| Solvent B | Acetonitrile |
| Sample | diluted propolis extract to 1:100 |
| | in 95% Et—OH |

### 2. Experiment Result

Through the experiments, the flavonoid components of the propolis extract were identified, and the content of each flavonoid component was analyzed, as shown in FIGS. 2 and 3. The flavonoid components identified were pinocembrin, quercetin, kaempferol, galangin, chrysin, caffeic acid, and p-Coumaric acid, and the content of each flavonoid component in propolis slightly varied by region. In particular, the content of each of pinocembrin, quercetin, galangin, and chrysin was found to be highest in most of the regions, and the highest quantitative content of flavonoid components was identified in propolis extracts obtained from Bonghwa (Bh), followed by Masan (Ms), Andong (Ad), and Gunwi (Gunwi).

### <Experimental Example 2> Determination of Effects of Independent Treatments of Different Concentration Levels of any One of Flavonoid Components, and Combined Treatments of any One of the Flavonoid Components with Sorbus commixta Ethanol Extract on LPS-Induced Inflammation

### 1. Experiment Method

### (1) Evaluation of Efficacy of Inhibiting Nitric Oxide (NO) Production

### \textcircled{1} RAW 264.7 Culture

RAW 264.7 cell line, a mouse macrophage cell line was purchased from KCLB (Korean Cell Line Bank, Korea). After RAW 264.7 was added with 10% fetal bovine serum (FBS, GIBCO, USA) and 1% antibiotics-antimycotic (GIBCO, USA), the cell was subcultured in Dulbecco's modified Eagle's medium (DMEM, WELGENE, Korea) in a 37° C., 5% CO₂ incubator (Thermo Fisher Inc., USA).

### \textcircled{2} Evaluation of Nitric Oxide (NO) Production

After RAW 264.7 cell line was independently treated with any one of the flavonoid componentsor Sorbus commixta extract, and was combinedly treated with any one of the flavonoid components and the Sorbus commixta extract, an evaluation of Nitric Oxide production (NO assay) was conducted to identify effects on NO secretion induced by LPS.

At this time, RAW 264.7 cells were seeded at 1×10⁵ cells/well in 96-well plates and cultured in a 37° C., 5% CO₂ incubator for 24 hours. Each cell was pretreated with samples by concentration (pinocembrin, quercetin, kaempferol, glangin, and chrysin at concentrations of 1, 2.5, 5, and 10 μg/mL, caffeic acid and p-Coumaric acid at concentrations of 2.5, 5, 10, and 25 μg/mL, and Sorbus commixta ethanol extract at concentrations of 25, 50, 100, and 250 ppm), respectively, and then the cells were treated with LPS (Sigma-Aldrich Inc, USA) to a final concentration of 100 ng/mL to induce the inflammatory response for 20 hours. After the induction of the inflammatory response, a 100 μL supernatant of a 96-well plate was added with a 100 μL Griess reagent (Sigma-Aldrich Inc., USA) at a concentration of 40 mg/mL (in water), and reacted in the dark for 15 minutes, and the optical density was measured at 540 nm using a Tecan infinite 200 microplate reader (Tecan Trading AG, Switzerland). A quantitative line was drawn using Sodium nitrite (Sigma-Aldrich Inc., USA), and each value of optical density was substituted to the quantitative line to determine the amount of produced Nitric Oxide (NO).

### 3 Cell Viability Analysis (MTT Assay)

A cell viability rate was analyzed by carrying out an MTT assay in order to determine the toxicity of the flavonoid components and Sorbus commixta ethanolic extract on the cell line treatment. The RAW 264.7 cell line was seeded 5×10⁴ cells/well in a 96 well plate, and cultured in a 37° C., 5% CO₂ incubator. After the culture, the flavonoid components and Sorbus commixta ethanolic extract were treated and additionally treated for the next 24 hours. After the additional culture, a 0.1 mg/mL MTT solution (Sigma-Aldrich Inc., USA) was dispensed in each well, the cells were cultured for 4 hours before the supernatant was removed. The resulting formazan was dissolved by seeding 100 μL of DMSO (Sigma-Aldrich Inc., USA) into each well, followed by orbital shaking (87.9 rpm) for 15 minutes, and optical density was measured at 540 nm using a Tecan infinite 200 microplate reader. The measured values of optical density were described as a percentage by substituting into the equation below.

Cell viability (%)=(Sample group O.D540/Control group O.D540)×100

### \textcircled{4} Statistical Analysis

Statistical comparisons of the present experiment results were analyzed using SPSS version 21 (SPSS Inc., USA). One-way ANOVA (analysis of variance) and Tukey's HSD test were also used to compare the significance of each experimental group. The statistical significance was tested at the p<0.05 level. All experimental results are expressed as mean±S.D.

### 2. Experiment Result

\textcircled{1} Measurement of RAW 264.7 Viability when Independently Treated with any One of Flavonoid Components or Sorbus commixta Ethanolic Extract Alone

To determine the cell viability of the RAW 264.7 cell line when independently treated with any one of the flavonoid components or the Sorbus commixta ethanol extract, cells were treated at concentrations of 1 μg/mL to 100 μg/mL for each flavonoid component, and 25, 50, 100, 250, and 500 ppm for Sorbus commixta ethanol extract. The results, as shown in FIG. 4, vary depending on the flavonoid components, but a greater than 80% cell viability rate was identified at a concentration of 10 μg/mL for pinocembrin, quercetin, kaempferol, galangin, and chrysin, and at a concentration of 25 μg/mL for caffeic acid and p-Coumaric acid.

In addition, greater than or equal to 80% cell viability rate was identified at a concentration of less than or equal to a 250 ppm Sorbus commixta ethanolic extract.

Therefore, in subsequent experiments, the concentrations shown above were set to the highest concentration at which the cells were treated.

\textcircled{2} Results of Measurement on the Amount of Nitrite Oxide Production when Independently Treated with any One of Flavonoid Components or Sorbus commixta Ethanolic Extract

In the inflammatory response of macrophages induced by LPS treatment, the treatment effects of any one of the flavonoid components or Sorbus commixta ethanol extract on NO production were determined.

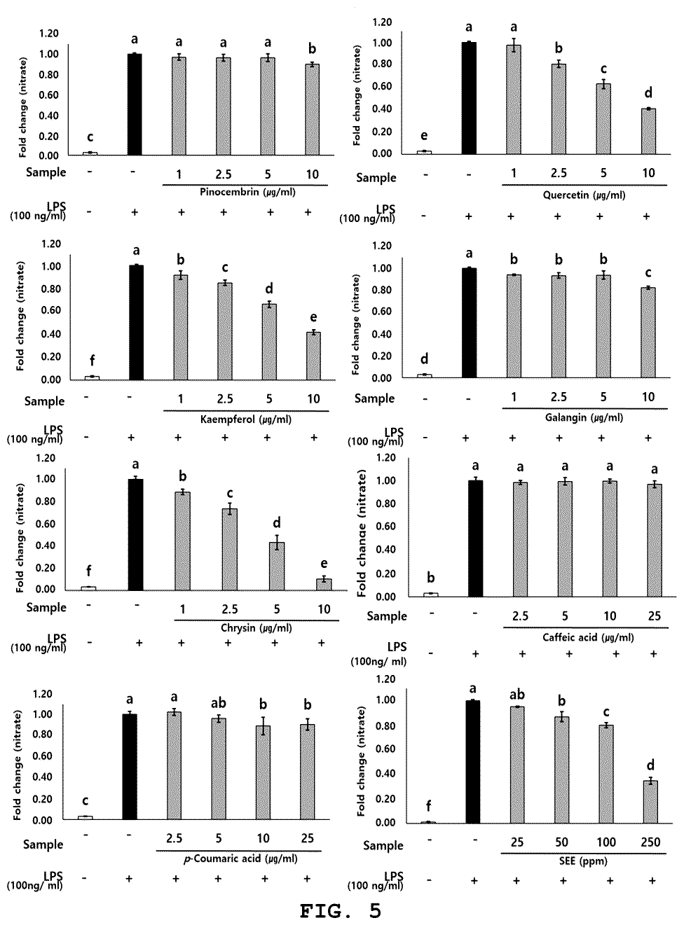

As a result, as shown in FIG. 5, when compared with each control, NO production rate was 90% and 82% at a concentration of 10 μg/mL of pinocembrin and galangin, respectively, and the rate was 97%, 90% at a concentration of 10 μg/mL of caffeic acid and p-Coumaric acid. Quercetin, kaempferol, and chrysin had the highest inhibitory effect on NO production among the flavonoid components, with 41%, 42%, and 10% NO production rate, respectively, in the 10 μg/mL concentration groups.

A 17% NO production rate was found respectively in the 250 ppm concentration groups. Furthermore, all three flavonoid components and the Sorbus commixta ethanol extract, which showed the highest suppression of NO production, reduced NO production in a concentration-dependent manner.

Consequently, suppression of NO production driven by 3 out of 7 components of propolis extract was determined. By utilizing the result into the subsequent experiments, synergetic effect of combined treatment of the flavonoid components and the Sorbus commixta ethanol extract on suppression of NO production was analyzed.

\textcircled{3} Measurement of NO Production when Combinedly Treated with any One of Flavonoid Components and Sorbus commixta Ethanol Extract

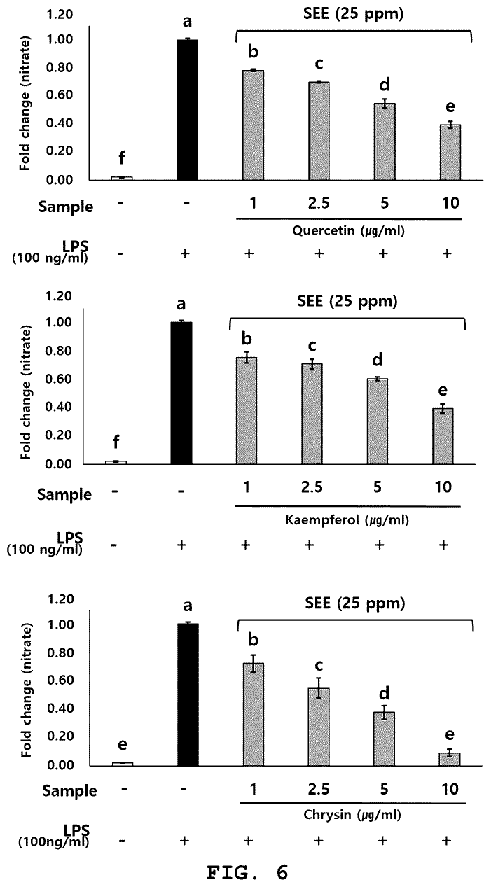

As shown in FIG. 6, independent treatment of flavonoid components resulted in a concentration-dependent increase in 97, 81, 63, and 41% NO production for quercetin, 92, 85, 66, and 42% for kaempferol, 89, 74, 43, and 10% for chrysin, and 95% for Sorbus commixta ethanol extract at a concentration of 25 ppm compared to the control. The combined treatment of flavonoid components and the Sorbus commixta ethanol extract resulted in NO production of 78, 70, 55, and 39% for quercetin, 75, 71, 60, and 39% for kaempferol, and 72, 55, 38, and 9% for chrysin. These results indicate that the combined treatment of flavonoid components and the Sorbus commixta ethanol extract resulted in a higher NO production suppression than the sum of the respective suppression rates, suggesting that the combination of any one of flavonoid components and the Sorbus commixta ethanol extract has a synergistic effect on suppressing NO production.

### <Experimental Example 3> Effects of Flavonoid Components and Sorbus commixta Ethanolic Extract on Allergic Response

Effects of quercetin, kaempferol, chrysin, and Sorbus commixta ethanolic extract on allergic response were determined by measuring the release of beta-Hexosaminidase through degranulation caused by the antibody-antigen binding in the RBL-2H3 basophilic cells.

### 1. Experiment Method

### (1) Analysis of Inhibiting Efficacy Against Degranulation of Beta-Hexosaminidase

### \textcircled{1} RBL-2H3 Culture

The RBL-2H3 basophilic cell line of Rat was purchased from ATCC (American Type Culture Collection, USA), and then it was added with a 10% fetal bovine serum and a 1% antibiotics-antimycotic in Dulbecco's modified Eagle's medium, and was subcultured in a 37° C., 5% CO₂ incubator.

\textcircled{2} beta-Hexosaminidase Release Assay

A beta-Hexosaminidase release assay was carried out to figure out effects of independent treatment of any one of the flavonoid components or the Sorbus commixta ethanolic extract, and combined treatment of any one of the flavonoid components (quercetin, kaempferol, chrysin) with Sorbus commixta ethanolic extract on the release of beta-Hexosaminidase through the degranulation of RBL-2H3 basophilic cell line. The RBL-2H3 cells were seeded at 8×10⁴ cells/well in the 96 well plates, and stabilized in a 37° C., 5% CO₂ incubator for 3 hours, then treated with anti-DNP-IgE (Sigma-Aldrich Inc., USA) to a final concentration of 250 ng/mL for cell sensitization, and incubated for 24 hours. After culturing, the supernatant liquid was removed and washed twice with Siraganian buffer (NaCl 119 mM, KCl 5 mM, Glucose 5.6 mM, PIPES 25 mM, MgCl₂, 0.4 mM, CaCl₂ 1 mM, BSA 0.1%, pH 7.2, Biosolution, Korea), and concentration-specific samples dissolved in Siraganian buffer were pretreated into the cells for 1 hour. After the pretreatment, the final concentration of DNP-BSA (Sigma-Aldrich Inc., USA) was treated to be at 150 ng/mL, and cultured for 1 hour to induce degranulation. After degranulation induction, 80 μL of supernatant and 20 μL of 10 mM 4-Nitrophenyl N-acetyl-β-D-glucosaminide (in 0.1 M citrate buffer, pH 4.5, Sigma-Aldrich Inc., USA) were reacted in the 96 well plate for 4 hours at a 37° C. incubator. After 4 hours, the reaction was terminated by adding 100 μL of 0.5 M sodium carbonate, and the optical density was measured at 405 nm using a Tecan infinite 200 microplate reader.

### 3 Cell Viability Analysis (MTT Assay)

Cell viability was analyzed by carrying out an MTT assay in order to identify toxicity driven by the treatment of the flavonoid components and Sorbus commixta ethanolic extract on each cell line. RBL-2H3 cells were seeded 5×10⁴ cells/well in each 96 well plate, and cultured in a 37° C. 5% CO₂ incubator for 24 hours. After culturing, the flavonoid components and Sorbus commixta ethanolic extract were treated into the cell plates and additionally cultured for 24 hours. After the additional culturing, a 0.1 mg/mL MTT solution (Sigma-Aldrich Inc., USA) was treated into the each well, and the cells were cultured for 4 hours, and then the supernatant liquid was removed. The resulting formazan was dissolved by seeding 100 μL of DMSO (Sigma-Aldrich Inc., USA) into each well, followed by orbital shaking (87.9 rpm) for 15 minutes, and optical density was measured at 540 nm using a Tecan infinite 200 microplate reader. The measured values of optical density were expressed as a percentage by substituting into the equation below.

$$Cell viability ( \% ) = ( Sample group O . D 540 / Control group O . D 540 ) \times 100$$

### \textcircled{4} Statistical Analysis

Statistical comparisons of the results of this experiment were analyzed using SPSS version 21 (SPSS Inc., USA). One-way ANOVA (analysis of variance) and Tukey's HSD test were also used to compare the significance of each experimental group, and statistical significance was tested at the p<0.05 level. All experimental results are expressed as mean±S.D.

### 2. Experiment Result

\textcircled{1} Measurement of RBL-2H3 Viability when Treated with any One of Flavonoid Components or Sorbus commixta Ethanolic Extract

To determine the cell viability of RBL-2H3 when treated with any one of the flavonoid components and Sorbus commixta ethanol extract, cells were treated with quercetin at concentrations of 2.5, 5, 10, and 25 μg/mL, and with different concentration levels of kaempferol and chrysin each.

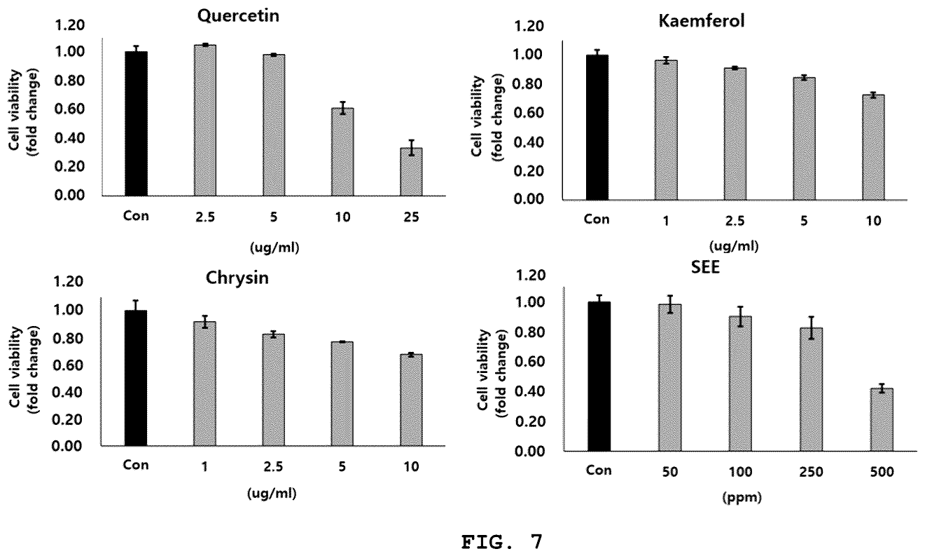

As a result, as shown in FIG. 7, an 80% or more cell viability was found at a concentration of 2.5, 5 μg/mL of quercetin, and at a concentration of 1, 2.5, 5 μg/mL of kaempferol, and at a concentration of 1, 2.5 μg/mL of chrysin. In addition, an 83% cell viability was found at a concentration of 250 ppm or less Sorbus commixta ethanolic extract. Therefore, in subsequent experiments, proper concentrations were set within the range of the concentrations mentioned above at which the cells were treated.

\textcircled{2} Effects of RBL-2H3 Degranulation when Independently Treated with any One of Flavonoid Components or Sorbus commixta Ethanolic Extract

First, RBL-2H3 cells' degranulation induced by antibody-antigen binding was determined when independently treated with any one of the flavonoid components or Sorbus commixta ethanolic extract. RBL-2H3 cells were sensitized with antibodies for one day, pretreated with different concentration levels of 3 selected flavonoid components, or Sorbus commixta ethanol extract for one hour, followed by additionally being treated with antigens. A group treated with neither the extract nor the antigen was a negative control, and a group independently treated with ethanol as the solvent for the sample and induced for degranulation only was a positive control. When RBL-2H3 cell degranulation is induced by antibody-antigen binding, beta-Hexosaminidase, an indicator of early response is released. On top of that, a substrate addition (P-NAG) leads to the degradation of the substrate and the suspension of the degradation reaction, resulting in a color change. In that case, the amount of beta-hexosaminidase may be determined by measuring the optical density, which may it possible to compare the degree of induced inhibition or induced activation of degranulation.

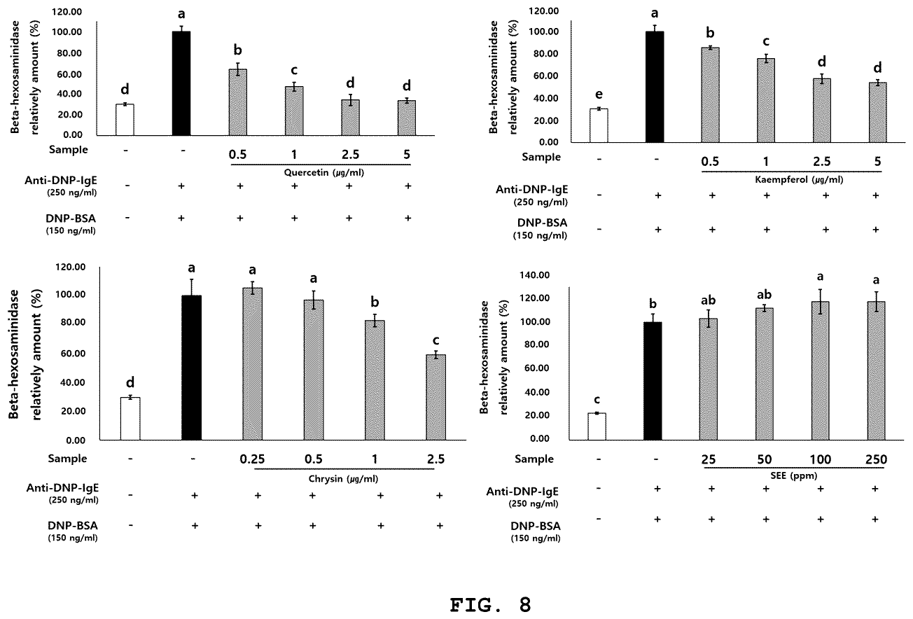

As a result, as shown in FIG. 8, compared to the positive control, quercetin and kaempferol released 64% and 85% of beta-Hexosaminidase, respectively, at a minimum concentration of 0.5 μg/mL, and chrysin released 83% of beta-Hexosaminidase at a concentration of 1 μg/mL, identifying concentration-dependent release inhibition which means a more release inhibition at a higher concentration.

However, in the group treated with the Sorbus commixta ethanol extract, an increase in the amount of beta-Hexosaminidase release from the minimum concentrations of 25 and 12.5 ppm of the Sorbus commixta ethanol extract was identified, respectively, with higher concentrations of the Sorbus commixta ethanol extract inducing more degranulation.

\textcircled{3} Effects on RBL-2H3 Degranulation when Combinedly Treated with any One of Flavonoid Components and Sorbus commixta Ethanolic Extract

The changes in the amount of beta-Hexosaminidase released by the combined treatment of the Sorbus commixta ethanolic extract with any one of the three selected active ingredients of the flavonoid components were intended to be determined. A group treated with neither the extract nor the antigen was a negative control, and a group independently treated with ethanol as the solvent for the sample and induced for degranulation only was a positive control.

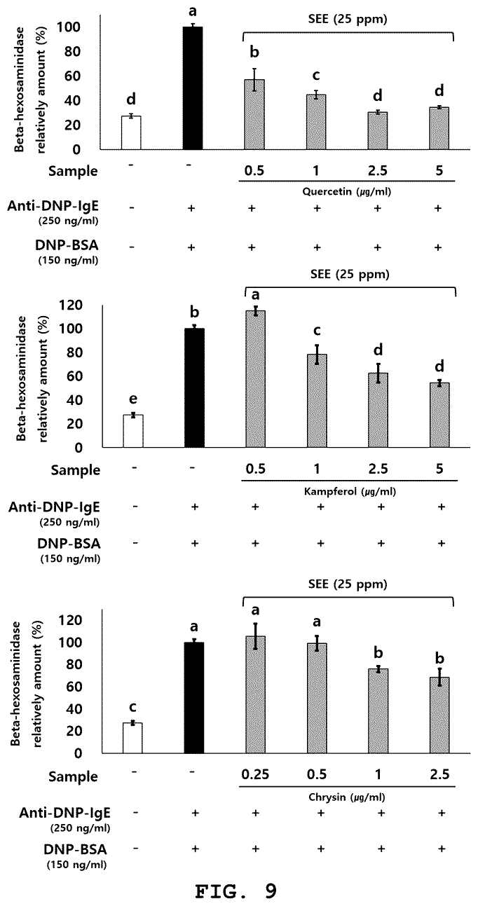

As a result, as shown in FIG. 9, in the combined treatment group with the Sorbus commixta ethanol extract and any one of the 3 selected flavonoid components, compared to the control group, quercetin at a concentration of 0.5 μg/mL, kaempferol at 2.5 μg/mL, and chrysin at 1 μg/mL which were respectively mixed with 25 ppm of Sorbus commixta ethanol extract, the production rate of beta-Hexosaminidase was 86.8%, 79.8%, and 95.9%, respectively, and the production rate of beta-Hexosaminidase gradually decreased with increasing concentration of the flavonoid components.

Previously, an increase in the amount of beta-Hexosaminidase release when independently treated with the Sorbus commixta ethanol extract was determined. However, this experiment suggests that when the Sorbus commixta ethanol extract was combinedly treated with any one of the flavonoid components, the flavonoid components appeared to inhibit degranulation induction by the Sorbus commixta ethanol extract, and thus may inhibit allergic reactions.

As may be seen, the synergistic effects of the combined treatment with the propolis ethanol extract and Sorbus commixta ethanol extract against the inflammatory responses and allergic responses are exhibited so that it was determined the extracts may provide a pharmaceutical composition and food composition for alleviating or treating inflammatory and allergic diseases as active ingredients.

### <Formulation Example 1. Preparing Pharmaceutical Tablets>

200 mg of a 1:25 by weight mixture of the propolis ethanol extract and Sorbus commixta ethanol extract prepared in Embodiment 1 was mixed with 175.9 g of lactose, 180 g of potato starch, and 32 g of colloidal silica. To this mixture was added a 10% gelatin solution, which was then ground and passed through a 14 mesh sieve. The passed mixture was dried, and the mixture, which was obtained by adding 160 grams of potato starch, 50 grams of talc, and 5 grams of magnesium stearate to the dried mixture, was tableted.

### <Formulation Example 2. Preparing Health Functional Foods>

According to Embodiment 1, 1 to 10 g of an extract prepared by mixing the propolis ethanol extract and Sorbus commixta ethanol extract in a 1:25 weight ratio was dissolved in 1 L of hot or membrane-filtered water and then was added with such food additives as nicotinamide of 0.001% to 5 by % weight, asparagine of 0.01% to 5% by weight, calcium citrate of 0.01% to 1.0% by weight, honey of 0.01% to 3% by weight, dextrin of 0.01% to 3% by weight, ginseng extract of 0.01% to 10% by weight, apple of 0.01% to 5% by weight, apple flavor of 0.05% to 1% by weight, fructooligo saccharide of 0.1% to 10% by weight, citric acid of 0.05% to 5% by weight, polysorbate of 0.01% to 3.0% by weight, benzoic acid of 0.05% to 1% by weight to make the food additives range from 0.28% to 55% by weight in the total composition, homogenized in a homogenizer, sterilized at 98° C. for 30 seconds, filled in a container at 86° C., and sealed. The sealed container was heated to sterilize the filling inside the container and was cooled at 30° C., manufacturing a health functional drink.

The description of the present disclosure has been made with reference to preferred embodiments, and the ordinarily skilled in the art to which this disclosure pertains will implement embodiments in the different form from the detailed description within the scope of the essential technical idea of the present disclosure. The scope of the essential technical idea of the present disclosure is described in the appended claims, all of the differences within the scope consistent with the technical idea of the present disclosure should be construed to be included in the present disclosure.

What is claimed is:

1. A pharmaceutical composition for alleviating and treating inflammatory diseases and allergic diseases, the pharmaceutical composition comprising a propolis ethanolic extract and a Sorbus commixta ethanolic extract.

2. The pharmaceutical composition of claim 1, wherein the propolis ethanolic extract comprises 0.5 to 2.5 μg/mL of Quercetin, 1 to 5 μg/mLof Kaempferol, and 1 to 2.5 μg/mLof Chrysin among flavonoid components.

3. A method of preparing a pharmaceutical composition for alleviating and treating inflammatory diseases and allergic diseases, the method comprising:
Preparing a propolis ethanolic extract by subjecting a propolis powder to an extraction process using an aqueous ethanol solution and performing filtration; and
preparing a Sorbus commixta ethanolic extract by subjecting a Sorbus commixta powder to an extraction process using an aqueous ethanol solution and performing filtration; and
mixing the propolis ethanolic extract and the Sorbus commixta ethanolic extract to produce the pharmaceutical composition.

4. The method of claim 3, wherein the propolis ethanolic extract comprises 0.5 to 2.5 μg/mLof Quercetin, 1 to 5 μg/mL of Kaempferol, and 1 to 2.5 μg/mLof Chrysin among flavonoid components.

5. A pharmaceutical composition for alleviating and treating inflammatory diseases and allergic diseases, the pharmaceutical composition prepared by the method of claim 3.

6. A food composition for alleviating and treating inflammatory diseases and allergic diseases, the food composition comprising a propolis ethanolic extract and a Sorbus commixta ethanolic extract.

7. The food composition of claim 6, wherein the propolis ethanolic extract comprises 0.5 to 2.5 μg/mL of Quercetin, 1 to 5 μg/mL of Kaempferol, and 1 to 2.5 μg/mL of Chrysin among the flavonoid components.

8. A method of preparing a food composition for alleviating and treating inflammatory diseases and allergic diseases, the method comprising:
preparing a propolis ethanolic extract by subjecting a propolis powder to an extraction process using an aqueous ethanol solution and performing filtration; and
preparing a Sorbus commixta ethanolic extract by subjecting a Sorbus commixta powder to an extraction process using an aqueous ethanol solution and performing filtration; and
mixing the propolis ethanolic extract and the Sorbus commixta ethanolic extract to produce the food composition.

9. The method of claim 8, wherein the propolis ethanolic extract comprises 0.5 to 2.5 μg/mL of Quercetin, 1 to 5 μg/mL of Kaempferol, and 1 to 2.5 μg/mL of Chrysin among the flavonoid components.

10. A food composition for alleviating and treating inflammatory diseases and allergic diseases, the composition prepared by the method of claim 8.
